(19) United States
(12) Patent Application Publication
(10) Pub. No.: US 2004/0170579 A1
(43) Pub. Date: Sep. 2, 2004

(54) Novel stabilising light-protection and skincare agents containing stabilised light-protection components and reduction of damaging light products

(76) Inventor: Dietmar Mobius, Gottingen (DE)

Correspondence Address: Michael C Barrett, Fulbright & Jaworski, Suite 2400, 600 Congress Avenue, Austin, TX, 78701, US

(21) Appl. No.: 10/474,010

(22) PCT Filed: Apr. 5, 2002
(86) PCT No.: PCT/DE02/01253

(30) Foreign Application Priority Data
Apr. 6, 2001 (DE) 101 17 336.9

(51) Int. Cl. A61K 7/42

(52) U.S. Cl. 424/59

(57) ABSTRACT

The invention relates to sun protecting agents and skin protecting agents containing (a) a light-protecting/sun protecting components (LSK) and (b) an energy or electron acceptor the spatial distance between (a) and (b) is not more than 10 nm if (b) is an energy acceptor. The spatial distance between (a) and (b) is not more than 3 nm if (b) is an electron-acceptor.

The present invention relates to the use of particular substances in photo protection agents and skin care products. These substances are suitable nanoparticles exhibiting a modified surface (NPMS) on the one hand and so-called super molecules on the other hand. In such super molecules are several photo protection molecules either directly or through a backbone portion covalently attached to one of more acceptor molecules. The present invention further relates to photo protection agents and skin care products comprising such substances.

Sun protection products and skin care products comprise so called photo protection components (PPCs) absorbing the radiation in the near UV and in the visible range, thereby reducing the negative effects of sun radiation on the skin. An example for a PPC and a photo protection molecule (PPM), respectively is the group of the cinnamic acid ester (H₅C₆—CH═CH—COOR, wherein the phenyl residue may be further substituted). A typical representative of this group is 3-(4-methoxyphenyl)-2-propenoic acid-2-ethylhexyl ester (p-CH₃O—C₆H₄—CH═CH—COO—CH₂—CH(C₂H₅)—CH₂—CH₂—CH₂—CH₃), also termed OMC (which stands for octylmethoxy-cinnamate). Upon absorption by these esters of the UV radiation and of light in the visible range, molecules in an electronically excited state are formed, which molecules may be deactivated by various means, or they may react photochemically. Amongst others, skin-damaging products, in particular radicals, are formed which have the effect that the photo protection components eventually become ineffective.

Further known additional components of photo protection agents and skin care products are TiO₂ and ZnO, which are water-soluble and, when applied externally, non-toxic substances (besides the cinnamic acid esters as PPCs). TiO₂ and ZnO, as used in photo protection agents and skin care products nowadays, that is, as free nanoparticles, not at all, or at least not sufficiently, capable to prevent the formation of damaging photo products such as radicals (i.e., only equipped with anti-coagulating means). Accordingly, these substances are not capable to add to a reduction of the damages of the skin by means of photo products and to a stabilization of the respective PPC in the sun protection product or skin care product, nor are they capable to contribute that the respective PPC remains effective as a UV-absorbing means over an extended period of time. In particular, radicals as photo products may elicit mutations in skin cells, thereby contributing to the generation of skin cancer.

As mentioned previously, the sun protection and skin care products thus far known suffer from the drawback that the consumer sunbathing over an extended period of time has to oil himself with the sun protection product again and again in order to protect the skin from the damaging UV-radiation by an active PPC.

Accordingly, the present inventor faced the problem to provide a new class of sun protection and sun care products, the PPC(s) of which (which is/are known per se) has/have been modified such that it/they exhibit/s an increased UV stability on the one hand (such that the sun protection product may be applied to the parts of the body exposed to the sun only in larger intervals), and that the formation of skin damaging photo products is reduced on the other hand, thereby reducing not only the aging process of the skin but also the risk to be taken ill with skin cancer.

That object has been solved by the present inventor by providing the sun protection product and skin care product as defined in the attached claims. The inventor proceeded from the assumption that the damaging effects exerted by the absorbed light energy on the skin and the photo protection component/the photo protection molecule can be significantly reduced by an energy or electron transfer (from the photo protection component that is electronically excited following light absorption) to a suitable energy and electron acceptor, respectively.

The sun protection and skin care products according to the present invention thus comprise, in addition to the PPC (e.g., a cinnamic acid ester such as OMC) an energy and electron acceptor, respectively, said acceptors, when combined with the PPC, working as an energy and electron transfer system, respectively. These energy and electron transfer systems are NPMS, in particular metal nanoparticles exhibiting a modified surface (MNPMS). Representatives thereof are Monolayer Protected Cluster Molecules (MPC molecules) and Monolayer Protected Alloy Cluster Molecules (MPAC molecules), as defined in ref. 6; semi-conductor nanoparticles exhibiting a modified surface (SCNPMS); dye nanoparticles exhibiting a modified surface (DNPMS); or super molecules.

All these transfer systems are characterized in that a molecule, generally several molecules, however, of a PPC is/are bound (chemically or physically) to one nanoparticle (e.g., of a dye, of a metal, or of a semi-conductor), or in case of a super molecule, is/are bound covalently to one or more acceptor molecules, either directly or via a backbone portion. In a preferred embodiment according to the present invention this brings about that one acceptor molecule can bind one or more photo protection molecules. However, even the reversed case is possible, that is, one photo protection molecule can bind one or even more acceptor molecules. Since an acceptor molecule can generally deactivate several excited photo protection molecules, the combination of one acceptor molecule and several photo protection molecules is clearly preferred according to the present invention.

In case of the super molecules (for an explanation of the term, see section “Definitions” further below), the chemical bonds present are generally covalent bonds.

The term “modified surfaces” as used herein means that the acceptor particles and molecules in sun protection and skin care products are in the form of being bound to PPCs, and form in combination with such PPCs forming the energy/electron transfer system. The bond may be a chemical or a physical bond, that is, a covalent bond, an ionic bond, a dipole-dipole interaction, van der Waals forces or hydrogen bridges as well as any combination of these interactions/bonds. Only in super molecules the bond is an exclusively covalent bond. Besides that, covalent bonds to the PPC are preferred also for the remaining acceptors.

### DEFINITIONS

Electron Acceptors are particles (atom clusters, nanoparticles, molecules) that are capable of taking up an electron from the excited state of the PPC and to re-transfer that electron (as a donor) to the PPC, wherein the acceptor will subsequently be in its original state again, thereby avoiding photochemical processes entailing damaging products.

Energy Acceptors are particles (atom clusters, nanoparticles, molecules) that are capable of taking up energy from the PPC and to convert same into heat, thereby avoiding photochemical processes.

Energy and Electron Transfer Systems denote the coupling between an energy/electron acceptor and one or more molecules of a PPC, wherein the coupling may be a physical or a chemical bond. These transfer systems are the prerequisite for a stabilized PPC in the sun protection product, because the PPC transfers its excitation energy/electron in the S₁-state (after uptake of solar energy) to the acceptor without triggering a photochemical reaction. Examples for energy and electron transfer systems according to the present invention are the above mentioned semi-conductor and dye nanoparticles exhibiting modified surfaces, the Monolayer Protected Cluster Molecules, the Monolayer Protected Alloy Cluster Molecules and the super molecules.

Photo Protection Component (PPC) is a molecule (a photo protection molecule), as it is comprised in sun protection products that are commercially available in order to filter out from sun light the UV A and B radiation and thus protect the skin from that radiation. According to the present invention, the term PPC is defined as any type of chemical compound absorbing the UV A and/or UV B radiation of the sun light, thereby protecting the human skin exposed to the sun from that radiation. Conventional PPCs are cinnamic acid esters, in particular 3-(4-methoxyphenyl)-2-propenoic acid-2-ethylhexylester (available as Eusolex® 2292 from Merck, Darmstadt, Germany). Another molecule, suitable for the absorption of UVA and B radiation and thus as PPC is octyltriazone (available as Uvinul® T150 from BASF, Ludwigshafen, Germany), 2,4,6-trianilino-p-(carbo-2′-ethylhexyl-1′-oxy)-1,3,5-triazine in its chemical designation. Further PPCs are listed in ref. 7, see “Chemische Sonnenschutzfilter”.

Nanoparticles (NPs) are characterized in that their diameter ranges from few nm to approximately 100 nm. According to a preferred embodiment, the diameter of the NP is no more than 20 nm. In particular, the diameter of the NP is no more than 10 nm. NPs consist of organic molecules (e.g., dye molecules), of metal atoms of an element (e.g., gold), or of several elements (e.g., alloy cluster) as well as of the components of inorganic semi-conductors (e.g., TiO₂, ZnO, SnO₂, WO₃, Sb₄O₆, ZrO₂ etc.) to give an example. Additionally, reference is made to refs. 11 and 12 as further literature regarding NPs.

According to the present invention, super molecules are all molecules in which an energy or electron acceptor as a core exhibits a shell of molecules of the PPC covalently attached thereto. An example for the core is a porphyrin backbone. In the alternative, the term “super molecule” also comprises a compound, in which an inert backbone molecule (e.g., glucose, bile acid, cyclodextrines, derivatives of adamantan, linear oligomers with, e.g., —OH or —NH₂ as side groups) comprises both an acceptor molecule and particle (or more) and a molecule or even more molecules bound to a PPC. Thus, the photo protection molecules are not directly bound to the acceptor molecule and particle but are bound through the backbone molecule (covalently).

Hereinafter, the expressions “photo protection agent” and “sun protection agent” will be used synonymously. The same is applicable for the expressions “photo protection component” and “sun protection component”.

However, what does the spatial vicinity of photo protection molecule (PPM) and energy and electron acceptor effect? Finally, it is crucial according to present invention that the light energy taken up by the PPC in the sun protection product does not entail the formation of radicals or other photo chemical reactions. Thus, the object resides in a reduction of the lifetime/half life of the electronically excited state of the PPC by reducing the distance between PPM and acceptor molecule to no more than 10 nm. Additionally, the acceptor molecule should have only a very short lifetime in its excited state. Provided both preconditions are met, the average time needed, e.g., by molecular oxygen to approach one of the excited molecules/particles by diffusion will be longer than the time needed for the excited molecule/particle to fall back into the ground state. On the one hand, this may be accomplished by the fact that the electronically excited PPM transfers energy to an energy acceptor. On the other hand, it may likewise be accomplished by the transfer of en electron from the electronically excited PPM to an electron acceptor. The emission of fluorescence of phosphorescence by the PPC is not a precondition for the transfer (of energy/electrons). The energy/electron transfer thus occurs, regardless whether the PPC will fluoresce, phosphoresce, or will do none of both. Quite conversely, it is important that the energy acceptor quickly converts the energy taken up into heat, thereby avoiding photochemical reactions. Likewise, it is important, that the electron acceptor quickly re-transfers the electron taken up to the ground state of the PPC, thereby avoiding photochemical reactions (bringing about damaging products, e.g., formation of radicals), the PPC itself returning into its original state.

As mentioned above, the use of TiO₂ in combination with PPCs in sun protection products has been described in the prior art. The use of TiO₂ (but also of other semi-conductor and metal or dye nanoparticles as well as super molecules) according to the present invention is based on the fact, that the PPMs are equipped, e.g., by means of functional groups, for an “attachment” to the acceptor particles and the acceptor molecules, respectively, which is the distinction to the state of the art. “Attachment” is to be understood to mean a chemical or physical bond (see the proceeding paragraph and, in particular, the last paragraph preceding the section “Definitions”). A consequence of the “attachment” is a significantly reduced distance between PPM and acceptor particle, which distance must—according to the invention—not exceed the threshold value of about 10 nm. Preferably, the distance is up to 5 nm or even only up to 3 nm. Particularly preferred values for this distance are 0.5 to 1 nm, 1 to 1.5 nm, 1.5 to 2 nm, 2 to 2.5 nm and 2.5 to 3 nm.

In case of covalent bonds, the size of the distance can be manipulated by modifying the spacer or linker, by which the covalent bond of the PPC to the nanoparticles occurs (this will be easily comprehensible when reading the description further below). Examples for linkers for MPC and MPAC molecules are thiol residues exhibiting distinct lengths (see ref. 6, scheme 1).

An exemplary and preferred embodiment for the attachment of cinnamic acid esters to TiO₂ and other semi-conductor nanoparticles resides in that the ester is provided with additional carboxy groups, which carboxy groups are suitable to allow an ionic interaction between the ester on the one hand and TiO₂ and the other semi-conductor nanoparticles, respectively, on the other. Further suitable semi-conductor nanoparticles according to the present invention are SnO₂-and ZnO₂ nanoparticles, that may correspondingly be “attached” to the ester.

According to a further preferred embodiment of the present invention MPC and MPAC molecules, for example surface modified, that is, with a monolayer PPC protected gold or other metal nanoparticles, are used for the energy and electron transfer. The coating occurs by means of so-called reactive groups (“self-assembly”), e.g., by means of thiols, in particular by the thiols listed in ref. 6, more particularly by the thiols listed in scheme 1 of ref. 6. Further reactive groups are disulfides.

Suitable cluster molecules (strictly speaking, such molecules are atomic and alloy clusters, respectively) according to present invention have a metallic core wherein the metal may in particular be a metal of groups Ib and VIII of the PSE (CAS version) as well as titanium. The noble metals are particularly suitable as a metallic core. Preferred metallic cores are the noble metals Au, Ag, Cu, Pt and Pd. Examples for the metallic core of the MPAC molecules are alloys, in particular alloys of the metal of the groups Ib and VIII of the PSE (CAS version), wherein the alloys of noble metals are particularly suitable. Preferred metallic cores of the MPAC molecules are the noble metallic alloys Au/Ag, Au/Cu, Au/Ag/Cu, Au/Pt, Au/Pd and Au/Ag/Cu/Pd.

The use of the PPC/acceptor system as NP is particularly advantageous according to the present invention. First, the form/size of nanoparticles allows the total mass of dye, semi-conductor and metal particles to be kept as low as possible. Second, the use of the nanoparticles allows to keep the amount of, e.g., noble metal (such as gold or platinum) to be kept low, which is a consequence of the highly dispersed distribution of the nanoparticles.

Energy Transfer and Energy Acceptors

Energy transfer competes with all processes of deactivation and reduces the lifetime of the electronically excited state (refs. 1 to 5). Any molecule having an absorption maximum of 20 to 50 nm longer than the absorption maximum (AM) of the primarily excited molecule, that is, the AM of the PPC, is suitable as an energy acceptor.

As mentioned previously, a remarkably reduced distance between PPM and acceptor particle, which must not exceed the threshold value of about 10 nm according to the present invention, is important to avoid the formation of damaging photo products. It is preferred that the distance in case of the energy transfer is no more than 5 nm or even no more than 3 nm. The better the overlap of the fluorescence band of the PPC and the absorption band of the energy acceptor, the larger the distance between both molecules/particles may be. An emission of fluorescence or phosphorescence by the excited PPC is not a precondition for the effect of the energy acceptor, however.

Such reduced distance of the molecules/particles is accomplished by the bond between PPC and energy acceptor, wherein the bond may be a chemical or a physical bond, that is, a covalent bond, an ionic interaction, a dipole-dipole interaction, van der Waals forces, or hydrogen bridges as well as any combination of these interactions/bonds. Covalent bonds of the acceptor to the PPC are preferred.

A PPC may, as an example, have an absorption maximum at 320 nm, a fluorescence maximum at 360 nm, an a phosphorescence maximum at 450 nm. A particularly suitable energy acceptor according to the present invention is thus a molecule/particle exhibiting an absorption band (strictly speaking, no maximum), as exemplified in FIG. 1. In other words, the acceptor should exhibit a strong absorption in the range of about 350 to 450/500 nm. These prerequisites are met by the initially mentioned MPC and MPAC molecules, in particular gold NPs.

Examples for energy acceptors having an extremely short lifetime in their excited state are non-fluorescing compounds having an absorption band in the near UV and blue range or the visible light. Compounds of the following dye classes meet such requirement: azo dyes, carotinoids, quinoid dyes, quinoline derivatives, coumarine dyes (partially), fluorescein and its derivatives, indigoid dyes, pyrene derivatives, triarylmethane dyes, xanthene dyes; porphyrins or porphyrin derivatives, phthalocyanins, anthraquinones, anthraquinone derivatives or mixtures of several of these dyes.

In an info sheet of the US Food and Drug Administration (FDA) of November 2000 (from the internet) “Summary of Color Additives Listed for Use in the Unites States in Foods, Drugs, Cosmetics, and Medical Devices”, various dyes with code names are listed as part 74, subpart C, for example, D&C Orange No. 4 (an azo dye); the chemical structures are listed in CTFA International Cosmetic Ingredient Dictionary, 4th edition, 1991 and 7th edition 1997 (CTFA=The Cosmetic, Toiletry, and Fragrance Association).

Electron Transfer and Electron Acceptors and Donors

In the alternative to the energy transfer, an electron may be transferred from the excited state of the primarily excited molecule (of the PPC, thus, e.g., of the cinnamic acid ester) to a suitable electron acceptor (electron transfer). A molecule or molecule cluster is suitable as an electron acceptor if it exhibits an empty or only singly occupied electron orbital that may take up the electron from the excited state of the PPC. For that, the energy of the orbital (on the physical scale with energy 0 for an electron in the vacuum) must be more negative than the energy of the orbital of the excited state of the PPC. It is required for the desired effect that an electron is re-transferred from the electron acceptor into the singly occupied ground state orbital of the PPC within extremely short time in order to suppress damaging secondary reactions (thus, the electron acceptor does simultaneously function as an electron donor). An electron acceptor/donor may be contemplated to be suitable according to the present invention, if (i) the orbital (S₁), that is to take up the electron from the excited S₁ state of the PPM is energetically lower than the S₁ orbital of the PPM and (ii) the S₀ orbital of the acceptor donating the electron to the PPM has a higher energy than the ground orbital (S₀) of the PPM. It is readily possible, however, that the electron that has been initially transferred from the PPC to an acceptor returns directly from there to the ground state orbital of the PPM. In such case, the prerequisite for an electron acceptor/donor according to the present invention is that its S₁ orbital, in terms of energy, is (as close to the middle as possible) between the S₀ and S₁ state of the PPC. Provided such prerequisite is met, and further provided the crucial distance of 3 nm between PPM and acceptor is kept and remains under such value, respectively, the half life of the excited state/radical is small, since the electron transfers from PPC to the acceptor and from S₁ of the acceptor to S₀ of the acceptor (in the alternative: of the PPM) occur very quickly.

Also in case of an electron transfer is a significantly reduced distance between PPM and acceptor, which distance must not exceed the threshold value of about 10 nm according to the present invention, important to avoid damaging photo products. As the electron transfer is the transfer of a corpuscle (which is distinct from the energy transfer), the distance between PPM and the electron acceptor is preferably no more than 3 nm, distances of up to 2 or only about 1 nm being particularly preferred. If the crucial distance between PPM and electron acceptor/donor is greater than 3 nm, the electrons can no longer be transferred from one molecule to the other, including the inability of the system to stabilize the PPC.

Likewise, all types of a binding interaction are suitable for the “attachment” of the electron acceptor/donor to the PPC: chemical and physical bonds, that is, covalentbonds, ionic directions, dipole-dipole interactions, van der Waals forces, hydrogen bridges as well as any combination of these interactions.

Suitable electron acceptors are semi-conductor nanoparticles. The conduction band of the semi-conductor functions as the electron acceptor. Its valence band from which an electron returns to the ground state of the PPC, functions as the donor. Almost simultaneously with such electron transfer from the valence band to the PPC occurs a transfer of an electron from the conduction band of the semi-conductor in its valence band. Alternative to that the electron transfer may also occur directly from the conduction band to the ground state of the PPC. It is important for the electron transfer to function that the ground and excited state of the PPC are energetically adapted to the energy of valence and conduction band of the semi-conductor (or vice versa).

Examples for electron acceptors, in addition to the already mentioned TiO₂, are SnO₂, ZnO, ZrO₂, WO₃ as semi-conductors as well as quinoid compounds as electron acceptors in super molecules, said compounds substantially exhibiting the acceptor qualities but not exhibiting the toxicity of di-octadecyl-4,4′-bipyridiniumperchlorate, mentioned below as a model system. Suitable electron acceptors are all molecules having a more positive reduction potential on the electrochemical scale than has the PPC. It is for this reason that the electron acceptor has to be adapted to the PPC (ample information and tables with redox potentials in refs. 8 and 17).

MPC/MPAC molecules may function both as energy and electron acceptors. The energy transfer has a greater crucial distance and is thus more efficient than the electron transfer, however.

Preparation of the Energy Electron Acceptor

Suitable energy/electron acceptors such as the MPC/MPAC molecules may be best prepared as described in refs. 6 and 9 to 12. The coating of the core occurs via a monolayer of the PPC, that is, e.g., of the 3-(4-methoxyphenyl)-2-propenoic acid-2-ester, the ethylhexyl residue of which has been replaced, e.g., with a short hydrocarbon chain with a least one thiol group. Likewise, the “attachment” of the PPC to the core metal may occur through disulfides (see ref. 9). A novel technique starts from dendrimers surrounding the nanoparticles formed (ref. 10). For that technique, suitable PPC dendrimers have to be prepared following known methods of synthesis (see refs. 18 and 19).

The preparation of super molecules consisting of PPC and acceptor molecule and, if required, a backbone portion occurs analogous to the synthesis of numerous super molecules that have been used to explore the electron transfer (dependency on energy and distance; see refs. 13 to 16). The single components are connected by distinct means, e.g., by amide or ester bonds. Furthermore, in case of conjugated systems. double bonds are formed between the components. Ether and thioether bonds are to be used as well. The backbone portion should provide well known reactive groups such as, e.g., —OH, 13 SH, —NH₂, —COOH. Examples for backbone portions are glucose, bile acid, cyclodextrin, glycerol, adamantan derivatives, oligomeric methacrylic acid, polyvinyl alcohol, polyallylamine, to the functional groups of which both the PPC and the acceptors may be bound by known methods. In the alternative, PPC and acceptor may be equipped with polymerizable and polycondensable groups and polymerized and polycondensated, respectively, to oligomers, if present in a suitable ratio.

The progress of such novel systems over the nowadays cosmetic preparations including nanoparticles that are added, e.g., for the reason of increasing the gloss of the preparation, resides in the targeted attachment to and thus a particularly strong interaction of the PPC with the acceptor (particle or molecule).

The concept to stabilize photo-protection agents and skincare products by energy and electron transfer and to reduce the formation of damaging photo products is the essential prerequisite according to present invention. The fundamentals of the energy and electron transfer have been amply investigated in systems of monomolecular layers (ref. 3). The novel photo protection agents and skincare products on the basis of energy transfer or electron transfer bring about a remarkable improvement over the conventional photo protection agents and skincare products, which is due to their increased light stability and reduced formation of damaging photoproducts. This allows a reduction of, e.g., the addition of antioxidants.

An electron acceptor, for the purpose of the present invention used only as a model due to its damaging side effects on organisms, which acceptor increases the light stability of a typical PPC, is di-octadecyl-4,4′-bipyridiniumperchlorate. In terms of energy, di-octadecyl-4,4′-bipyridinium-perchlorate is not optimal, however, which causes a non-optimal re-transfer of the electron into the ground state of the PPC. Accordingly, in this model system an optimal stabilization of the PPC could not be expected. Rather, that system was good to prove that electron transfer processes entail a stabilization of the PPC.

FIG. 1 depicts the absorption spectrum of a monolayer of gold nanoparticles, coated with octylthiol, on glass.

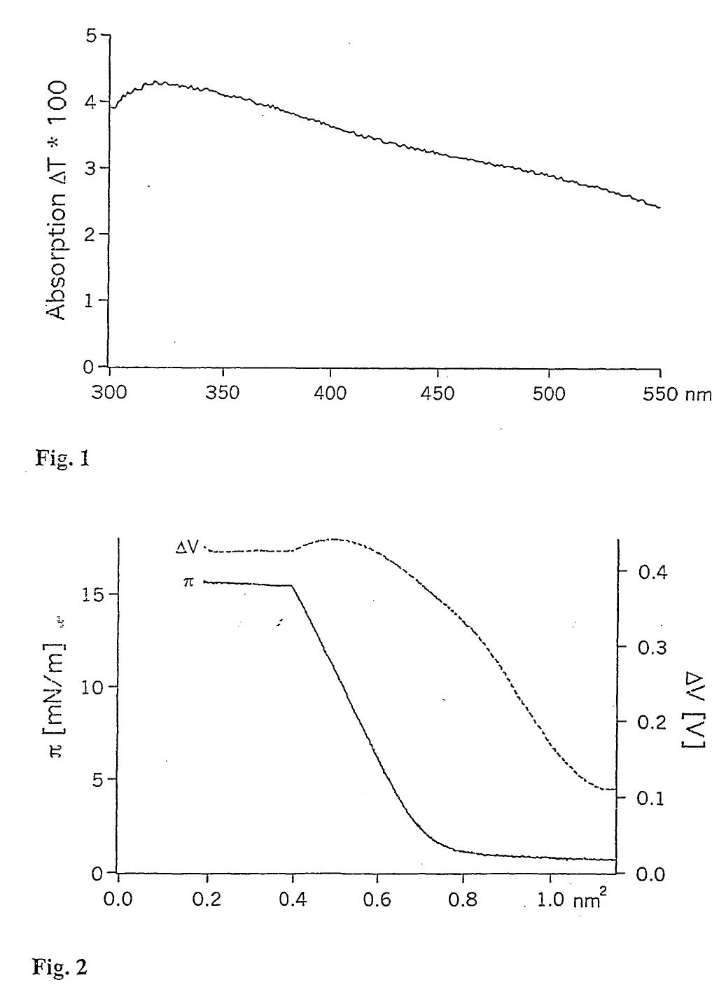

FIG. 2 describes the isotherms of a monofilm of Eusolex® 2292 (see Example 1) on water.

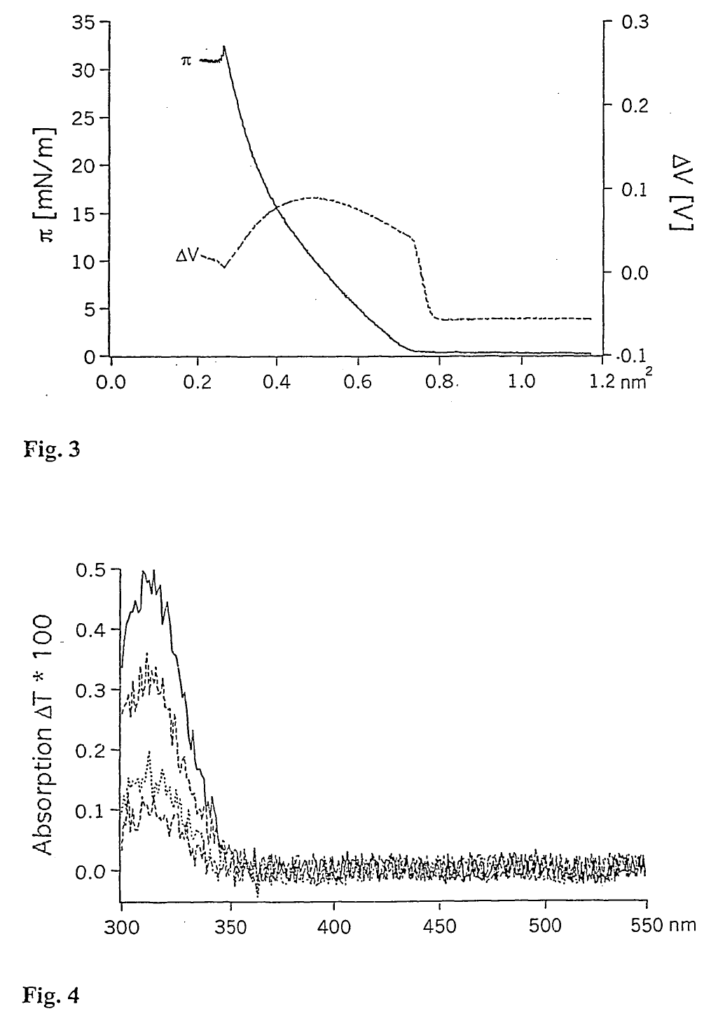

FIG. 3 depicts the isotherms of the mixed film of Eusolex® 2292:octadecylmalonic acid (OMA)=1:2 on water (see Example 2).

FIG. 4 depicts the absorption spectra of system A (see Examples 2 and 3) following distinct times of irradiation.

FIG. 5 represents the absorption spectra of system B (see Examples 2 and 3) following distinct times of irradiation.

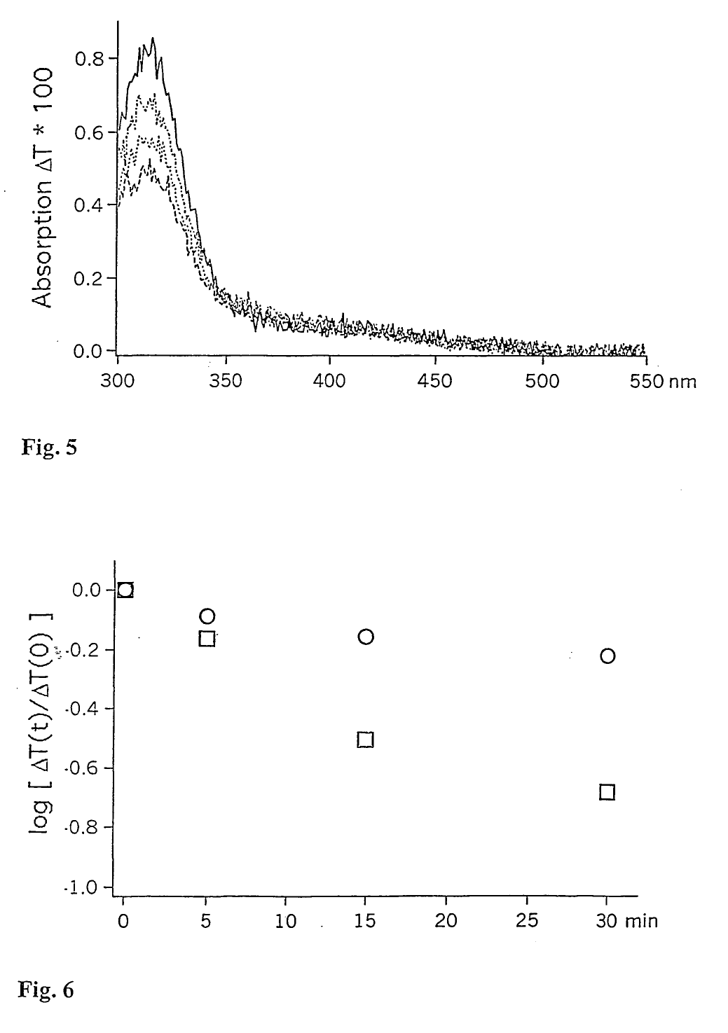

FIG. 6 is the evaluation of the absorption spectra at a wave length of 310 nm as depicted in FIG. 4 (squares) and 5 (circles).

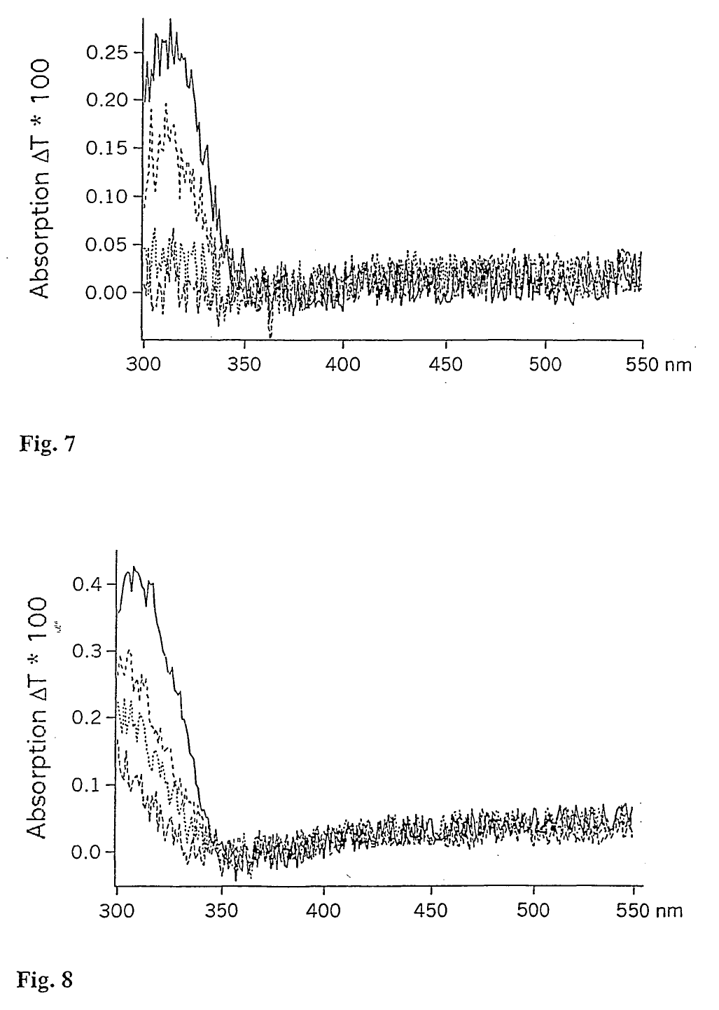

FIG. 7 depicts the absorption spectra of system C following distinct times of irradiation (see Examples 2 and 4).

FIG. 8 depicts the absorption spectra of system D following distinct times of irradiation (see Examples 2 and 4).

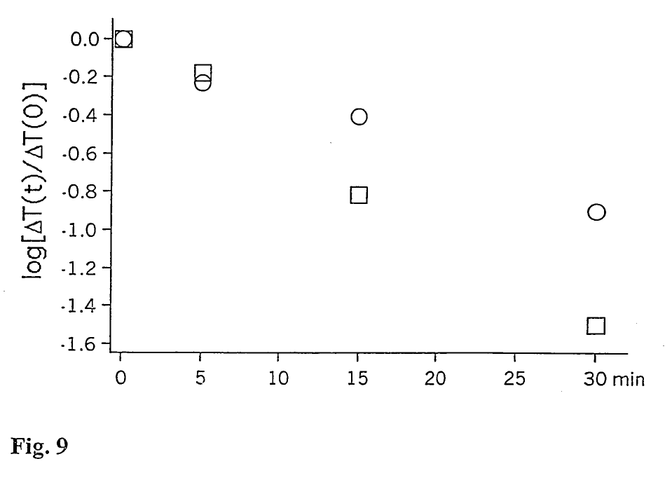

FIG. 9 is the evaluation of the absorption spectra at a wave length of 310 nm as depicted in FIG. 7 (squares) and 8 (circles).

The following examples demonstrate that the light stability of a typical photo protection component is increased by the presence of MPC and MPAC molecules in a distance of about 3 nm. This brings inevitably about also a reduction in the formation of damaging photo products.

### EXAMPLES

The increase in light stability of a cinnamic acid ester as the prototype of a PPC has been demonstrated in systems of monomolecular layers as a model. Such structures readily enable to arrange molecules in plain surfaces with a defined distance.

### Example 1:

Formation of Monomolecular Films

The ester (3-(4-methoxyphenyl)-2-propenoic acid-2-ethylhexyl ester, abbreviation EU), commercially available from Merck, Darmstadt, Germany as Eusolex® 2292, was used as a model substance. That product forms monomolecular films on water subsequent to spreading of a 10⁻³ M solution in chloroform, which films have been characterized by measurement of surface pressure/area (π/A) and surface potential/area (ΔV/A) isotherms at room temperature (FIG. 2).

Additionally, for the reason of a film transfer to solid supports mixed films of EU and octadecylmalonic acid (OMA), molar ratio EU:OMA=1:2, were formed on water by spreading of a mixed solution of the components and likewise characterized (FIG. 3). The curve of the π/A-isotherms indicates the formation of stable monofilms on water up to a surface pressure of π=15 mN/m (EU) and 30 mN/m (EU:OMA=1:2). In the Brewster angle microscope the monofilms of EU and EU:OMA=1:2 appear to be homogenous, up to the compression where the almost horizontal section is reached. After that, small round brighter domains are formed indicating the collapse of the film. These results constitute the prerequisites for the establishment of the model systems.

### Example 2:

Transfer of the Monofilms to Glass Plates, Establishment of the Model Systems

A transfer of the monofilms from the surface of water to glass plates was attempted to be achieved by a vertical dipping of the plates through the film at constant surface pressure (Langmuir-Blodgett technique). The transfer is registered by determining the decrease in area of the film on the surface of the water during the dipping process. The monofilms of EU could not be directly transferred to glass plates when applying a surface pressure of π=20 mN/m. Even a hydrophobizing of the glass plates by transferring a monofilm of eicosylamine (EA) at a surface pressure of π=40 mN/m prior to dipping the plate through the monofilm of EU was unsuccessful. Quite conversely, mixed monofilms of the molar composition EU:OMA=1:2 could be transferred both at a surface pressure of π=20 mN/m and at π=10 mN/m. A transfer occurred during the process of both dipping and removing. Between the dipping procedures the monofilm may be removed from the surface of water and replaced by another monofilm. These dipping procedures will be identified hereinafter by the arrows when describing the systems as established: ↓ denotes a transfer when dipping the glass plate through the film, ↑ correspondingly meaning a transfer when removing the glass plate again; in case of ↓↑ a layer is transferred both during the dipping and the replacement procedure. In this way, double layers will be formed. In an abbreviated form, the system is denoted as follows: system A: glass, ↑EA 40, ↓↑EU:OMA 1:2 10.

That means that the glass plate initially dipped in water is coated with a monolayer of eicosylamine (EA) at a surface pressure of 40 mN/m when removing the plate. Subsequently, the monofilm of EA is removed, a mixed film of Eusolex® 2292 and OMA in a molar ratio of 1:2 is formed and transferred to the glass plate both when dipping the glass plate and also when removing it again at a surface pressure of 10 mN/m. Following this procedure, system A is fully established.

Gold nanoparticles (abbreviated Au) were used as energy acceptor for the model substance. The nanoparticles were coated by reacting them with octylthiol following the method described in ref. 6. They likewise form monofilms on water that are transferred to hydrophobic glass plates at a surface pressure of π=10 mN/m only during the dipping procedure. In the course of removing them there is no transfer. In order to characterize the energy transfer from EU to Au following excitation, layer systems having the following sequence were constructed:

system B: Glas, ↑EA 40, ↓Au 10, ↑-, ↓↑TEU:OMA 1:2 10

In system B the gold nanoparticles are separated from the cinnamic acid ester by the layer of octyl residues on the gold and the long hydrocarbon chains of OMA and the substituents of EU. The distance is about 3 nm.

For the construction of model systems for the electron transfer, the electron acceptor di-octade-cyl-4,4′-bipyridiniumperchlorate (S135) was used. The dimethyl derivative is a strong toxin due to its capacity to block electron transfer processes in biological systems. Therefore, the dioctadecyl derivative is no more than a model and not suitable for the application in sun care products. The derivative is used in a mixed layer together with stearic acid (C₁₈) in a molar ratio of 1:10 as it turned out to be an excellent acceptor in investigations regarding the electron transfer (ref. 3). The systems investigated are:

system C: glass, ↑EA 40, ↓EU:OMA 1:2 10, ↑C₁₈ 20

system D: glass, ↑EA 40, ↓EU:OMA 1:2 10, ↑S135:C₁₈ 1:10 20

Only one layer of EU:OMA 1:2 was transferred and coated with a layer of stearic acid (C₁₈) in reference system C and with acceptor with the mixed layer S135:C₁₈ 1:10 in a system D. It is for this reason that the cinnamic acid ester (EU) and the electron acceptor S135 are at the same contact surface no more than about 0.5 nm apart from each other, only laterally and statistically seen.

The absorption spectra of these systems have been measured in a particular apparatus (see FIG. 7 to 9). The difference in transmission ΔT between a reference zone without the layer to be determined and a zone with such layer is determined. ΔT is proportional to the absorption of the layer for small values.

### Example 3:

Increase of the Light Stability by Energy Transfer

FIG. 4 depicts absorption spectra of a glass plate with system A before irradiation and following irradiation with white light of a 200 W Hg lamp with increasing times t of irradiation: 5 minutes, 15 minutes, 30 minutes. The absorption decreases remarkably during irradiation. As a comparison, FIG. 5 depicts corresponding absorption spectra of a glass plate with system B. The times of irradiation under the same conditions as in FIG. 4 are here: t=0; 5; 15 and 30 min. The comparison of FIG. 4 and FIG. 5 demonstrates directly that the light stability of EU in the presence of the layer of gold nanoparticles in a distance of about 3 nm is significantly increased.

### Example 4:

Increase of the Light Stability by Electron Transfer Processes

FIGS. 7 (system C without electron acceptor) and 8 (system D with electron acceptor) depict the absorption spectra before and following 5, 15 an 30 min irradiation with white light of a 200 W Hg lamp under otherwise identical conditions. The decrease of the absorption is remarkably reduced in system D as compared to system C, which is likewise demonstrated by the evaluation of FIG. 9 (analogous to FIG. 6).

FIG. 9 (the values from FIG. 8 were corrected by subtracting the value ΔT (S135)=0.06 at 310 nm) clearly demonstrates the increase of stability in the presence of the acceptor layer. It is true that stabilization is not as strong as in case of the energy transfer. However, in system D the optimal electron donor re-transferring the electron into the ground state of EU is lacking. The result demonstrates, though, that electron transfer processes are suitable to increase the stability of a PPC.

References:

1. Förster, Ann. Phys. (Leipzig) 2, 55 (1948)

2. Kuhn, J. Chem. Phys. 53, 101 (1970)

3. Kuhn and Möbius, in Investigations of Surfaces and Interfaces, Vol. IXB, 2^{nd} ed. (eds: Rossiter and Baetzold), John Wiley & Sons, Inc., New York, pages 375 pp.(1993)

4. Barth et al. in Optische Anregung organischer Systeme, Verlag Chemie, Weinheim, pages 639 pp. (1966)

5. Bücher et al., Mol. Cryst. 2, 199 (1967)

6. Templeton et al., Acc. Chem. Res. 33, 27-36 (2000)

7. Schauder, Schrader, Ippen, Göttinger Liste 1996, 4th edition, Blackwell Wissenschafts-Verlag, Berlin 1996, pages 40 pp.

8. A. J. Bard, H. Lund (editor): Encyclopedia of Electrochemistry of the Elements, Vol. XI, XII und XIV, Marcel Dekker, New York 1978 (XI, XII) and 1980 (XIV)

9. Porter, Ji, Westcott, Graupe, Czemuszewicz, Halas, Lee, Langmuir 14, 7378-7386 (1998)

10. Crooks, Zhao, Sun, Chechik, Yeung, Acc. Chem. Res. 34, 181-190 (2001)

11. Henglein, Ber. Bunsenges. Phys. Chem. 101, 1562-1572 (1997)

12. Härtl, Beck, Roth, Meyer, Hempelmann, Ber. Bunsenges. Phys. Chem. 101, 1714-1717 (1997);

13. Wasielewski, Chem. Rev. 92, 435-461 (1992)

14. Verhoeven, Gust, Moore, Adv. Photochem. 16, 1 (1992)

15. Barbara, Meyer, Ratner, J. Phys. Chem. 100, 13148-13168 (1996)

16. Kaifer, Kaifer, Supramolecular Electrochemistry, Wiley-VCH Weinheim 1999

17. CRC Handbook Series in Organic Electrochemistry, CRC Press, Boca Raton 1976 pp.

18. Buhleier, Wehner, Vögtle, Synthesis 155-158 (1978)

19. Newkome, Moorefield, Vögtle, Dendritic Molecules: Concepts, Syntheses and Perspectives; VCH, Weinheim 1996

1. Sun protection and skin care product, comprising
(a) a photo protection/sun protection component (PPC) and
(b) an energy or electron acceptor,
wherein the spatial distance between (a) and (b) is no more than 10 nm if (b) is an energy acceptor, and wherein the spatial distance between (a) and (b) is no more than 3 nm if (b) is an electron acceptor.

2. The product of claim 1, wherein (b) is an electron acceptor and the spatial distance between (a) and (b) is 0,5 to 1 nm, 1 to 1,5 nm, 1,5 to 2 nm, 2 to 2,5 nm or 2,5 to 3 nm.

3. The product of claim 1 or 2, wherein (a) is derived from a cinnamic acid ester, in particular from 3-(4-methoxyphenyl)-2-propenoic acid-2-ethylhexyl ester or another material listed in Table 2 of ref. 7 and denoted as a chemical sun filter, and wherein the cinnamic acid ester and the chemical sun filter, respectively, is a molecule substituted with a reactive group, in particular with a thiol or disulfide.

4. The product of any of the preceding claims, wherein (a) and (b) are connected with each other by at least one covalent bond.

5. The product of any of the preceding claims, wherein (b) is a metal nanoparticle, a semi-conductor nanoparticles or a dye nanoparticle.

6. The product of claim 5, wherein the semi-conductor nanoparticle is TiO₂, ZnO, SnO₂, WO₃, Sb₄O₆ or ZrO₂.

7. The product of claim 5, therein the dye nanoparticles are composed of molecules of an azo dye, a carotinoid dye, a quinoid dye, a quinoline derivative, a coumarin dye, fluorescein or one of its derivatives, an indigoid dye, pyrene derivatives, triarylmethane dyes, xanthene-dyes, porphyrin or a porphyrin derivative, a phthalocyanin, anthraquinone, an anthraquinone derivative or of molecules of several of these dyes.

8. The product of claim 5, wherein the metal nanoparticles are Au, Ag, Cu, Pt or Pd and the alloy is Au/Ag, Au/Cu, Au/Ag/Cu, Au/Pt, Au/Pd oder Au/Ag/Cu/Pd respectively.

9. The product of any of claims 1-4, wherein the molecule consisting of (a) and (b) is a super molecule, in which at least one PPM and at least energy or electron acceptor molecule are covalently attached to each other, either directly or via a backbone portion.

10. Use of (a) a photo protection/sun protection component (PPC) and (b) an energy or electron acceptor as common components in sun protection and skin care products, characterized in that the spatial distance between (a) and (b) is no more than 10 nm, if (b) is an energy acceptor, and wherein the spatial distance between (a) and (b) is no more than 3 nm, if (b) is an electron acceptor.
